(19) United States
(12) Patent Application Publication
(10) Pub. No.: US 2005/0273271 A1
(43) Pub. Date: Dec. 8, 2005

(54) Method of characterizing cell shape

(76) Inventors: Aibing Rao, Burlingame, CA (US); David M. Roof, San Carlos, CA (US); Mary Maxon, San Francisco, CA (US); Eugeni Vaisberg, Foster City, CA (US)

Correspondence Address: BEYER WEAVER & THOMAS LLP, P.O. BOX 70250, OAKLAND, CA, 94612-0250, US

(21) Appl. No.: 11/098,020

(22) Filed: Apr. 1, 2005

Related U.S. Application Data
(60) Provisional application No. 60/559,902, filed on Apr. 5, 2004.

(51) Int. Cl. G06F 19/00; G01N 33/48; G01N 33/50; C12Q 1/04

(52) U.S. Cl. 702/19; 435/34

(57) ABSTRACT

An image analysis method characterizes cells based on the elongation. It analyzes an image of the cells and automatically identifies points or locations of high contrast (i.e., regions where the transition from intense signal to weak signal occurs abruptly, over a short distance). Groups of contiguous high contrast points collectively define a putative cell edge. Identified edges are then analyzed to determine whether they are elongated. In one example, edges with relatively low curvature are deemed elongated. The curvature analysis may be accomplished by calculating a shape descriptor for each edge (e.g., calculating the circular variance for each edge). One or more stages of the analysis may employ some degree of filtering, smoothing or other processing to remove certain artifacts in the image.

### CROSS REFERENCE TO RELATED APPLICATIONS

This application claims priority under 35 USC § 119(e) from U.S. Provisional Patent Application No. 60/559,902, filed Apr. 5, 2004 and titled “METHOD OF CHARACTERIZING CELL SHAPE.” This application is related to the following US Patent documents: U.S. patent application Ser. No. 09/310,879 by Crompton et al., filed May 14, 1999 and titled “DATABASE METHOD FOR PREDICTIVE CELLULAR BIONINFORMATICS;” U.S. patent application Ser. No. 09/311,996 by Crompton et al., filed May 14, 1999 and titled “DATABASE SYSTEM INCLUDING COMPUTER FOR PREDICTIVE CELLULAR BIOINFORMATICS;” U.S. patent application Ser. No. 09/311,890 by Crompton et al., filed May 14, 1999 and titled “DATABASE SYSTEM FOR PREDICTIVE CELLUAR BIOINFORMATICS;” U.S. patent application Ser. No. 09/888,063 by Drubin et al., filed Jun. 22, 2001 and titled “IMAGE ANALYSIS FOR PHENOTYPING SETS OF MUTANT CELLS;” and U.S. patent application Ser. No. 10/621,821 by Kutsyy et al., filed Jul. 16, 2003 and titled “METHODS AND APPARATUS FOR INVESTIGATING SIDE EFFECTS”. Each of these references is incorporated herein by reference for all purposes.

### BACKGROUND

This invention relates to methods, computer program products, and apparatus for analyzing images of biological systems such as individual cells. More specifically, it relates to methods, computer program products, and apparatus that identify elongated edges of biological features in the images of biological features.

A number of methods exist for investigating the effect of a treatment or a potential treatment, such as administering a drug or pharmaceutical to an organism. Some methods investigate how a treatment affects the organism at the cellular level so as to determine the mechanism of action by which the treatment affects the organism.

One approach to assessing effects at a cellular level is to capture images of cells that have been subject to a treatment. A skilled researcher can visually inspect the images to make an assessment. More preferably, a computer based image analysis method makes the assessment. However, there is a need for reliable algorithms for analyzing image-derived data in order to accurately and reliably characterize the effects of a treatment at a cellular level.

In general, the overall shape of fungal cells such as yeast is very informative. Gross shape features indicate where the cell currently resides in the overall cell cycle. This is illustrated by the yeast cell cycle presented in FIG. 1. Further information about the yeast Saccharomyces cerevisiae cell cycle may be found in Hartwell 1981, “The Molecular Biology of the Yeast Saccharomyces cerevisiae,” Pringle J. R. and Hartwell, L. M., pp. 97-142, Cold Spring Harbor Laboratory Press, incorporated herein by reference. FIG. 1 shows, in cartoon fashion, a yeast cell progressing from a mitotic (M) state 103, through a G1 state 105, and an S state 107. The G2 state is not shown explicitly. Of note, the S state involves budding with formation of a projection 109. This grows to a nearly fully formed separate cell at the mitotic state 103.

If at any stage in the cell cycle, progression to the next stage is arrested the yeast cell will produce an elongated structure. This structure is represented in cell shapes 111, 113, and 115, each of which is shown on a different branch from the basic cell cycle. The elongated fungal cell 111 approximates a shape typically observed when the cell cycle is arrested at the mitotic stage. Similarly, elongated fungal cell 113 approximates a shape typically observed when the cell cycle is arrested at the G1 phase, and cell 115 approximates the corresponding shape for arrest at the S phase. Of course, the cell shapes depicted in FIG. 1 are rough approximations of the actual shapes observed and do not reflect the range of shapes observed at any stage, whether arrested or not.

Classifications based on elongation can be useful in various contexts. It would be useful to have image analysis methods that could quickly characterize a treatment based on a measure of the elongation of a population of cells such as fungal cells. In many contexts, this phenotypic trait could be used to suggest a particular mechanism of action, the potency of a treatment, etc.

The present invention provides methods and apparatus for characterizing cells, assessing the effects of treatments on cells, and specific algorithms for analyzing data derived from images of cells and cell components so as to characterize a population of cells based on measures and indications of the existence of elongated cells or cells having regions of elongation.

### SUMMARY

This invention characterizes one or more cells based on the elongation of those cells or a component thereof. It analyzes an image of the cell(s) and automatically identifies points or locations of high contrast (i.e., regions where the transition from intense signal to weak signal occurs abruptly, over a short distance). Groups of contiguous high contrast points collectively define putative cell edges, which are subsequently analyzed to determine whether they are elongated. In one example, edges with relatively low curvature are deemed elongated. The curvature analysis may be accomplished by calculating a shape descriptor for each edge (e.g., calculating the circular variance for each edge). One or more stages of the analysis may employ some degree of filtering, smoothing or other processing to remove certain artifacts in the image.

One aspect of the invention provides a method of characterizing cellular elongation in a population of cells. The method may be described by the following sequence: (a) receiving image data showing signal intensity versus position in an image of the population of cells (which was optionally treated); (b) determining values of a gradient in signal intensity at multiple positions in the image; (c) identifying edges of cells in the image from the values of the gradient determined in (b); (d) determining whether individual edges identified in (c) are elongated; and (e) characterizing the population of cells based on elongation. Preferably, the edges identified in (c) were identified without needing to first identify individual cells in the image by segmentation or other technique.

In some cases, it is desirable that the image of the population of cells was obtained when at least some of the cells were alive. Therefore, in certain cases, the image of the population of cells was obtained without staining or fixing the cells.

Various techniques can be employed to generate images showing the population of cells. For example, the image may be produced by a technique that captures contrast in absorbance, refractive index, etc. Specific examples of the microscopy techniques that can be employed in image capture include phase contrast microscopy, Hoffman modulation contrast microscopy, differential interference contrast microscopy, bright field microscopy, and the like.

To assess the effect of a treatment, one may use an elongation assay as described above on a test population and a control population in the following manner: (i) exposing the test population to a stimulus prior to producing the image of the population; (ii) analyzing the image to characterize the elongation in the test population (e.g., performing operations (a)-(e) above); (iii) imaging a control population of cells that has not been treated with the stimulus to characterize the elongation in the control population; and (iv) comparing the elongation in both populations of cells to gain information about the effect of the stimulus on the population of cells. Various stimuli may be analyzed in this manner. One of particular interest is exposure to a chemical compound such as a drug of drug candidate. A dose response signature of the compound may be obtained by repeating the image analysis and elongation characterization with the chemical compound at a concentration that is different from that used in an initial pass.

Various considerations come into play in determining values of the gradient in signal intensity. One of these is which spatial direction(s) in the image should be used to make the determination. For example, the gradient be measured in the vertical direction, the horizontal direction, a diagonal direction, some combination of these. In some embodiments, gradient values are obtained in each of two directions, which may be substantially orthogonal to one another (e.g., the vertical and horizontal directions).

Frequently it will be convenient to identify putative cell edges by selecting only those pixels having gradient values that are greater than a threshold value. The threshold value may be fixed for multiple images or may be calculated from the image data using a method such as an adaptive threshold technique. The thresholding technique may make use of histograms of pixel gradient values.

In some embodiments, edges are identified by large positive gradients and large negative gradients. Thus, edges are identified by abrupt changes in intensity from high to low and from low to high. Selecting pixels having gradient values that are greater than a threshold value may involve selecting not only pixels having positive gradient values that are greater than a positive value of the threshold but also selecting pixels having negative gradient values that are more negative than a negative value of the threshold.

A shape descriptor can be used for determining whether the individual edges are elongated. In one approach, this descriptor specifies a degree of curvature in an edge. In a specific embodiment, the shape descriptor is a circular variance in an edge. To make the ultimate determination of whether an edge is elongated or not, the method may compare the values of the shape descriptor to an elongation threshold. Further, the final characterization of the cells may involve determining a fraction of edges or edge pixels in the image that are determined to be elongated.

The present invention has many applications. In one preferred application discussed herein, the population of cells comprises fungal cells. In this application, the invention may be used to classify a treatment of the fungal cells based on, for example, its ability to arrest progress through the cell cycle. Frequently, the treatment in question will be contact with a known or putative anti-fungal agent. In another example, the treatment is a genetic manipulation of the fungal cells. In another example, the treatment is contact with an agent that induces differentiation, such as mammalian serum which induces a switch to filamentous growth.

A specific method for characterizing elongation in a fungal cells may be described by the following sequence: (a) receiving image data showing signal intensity versus position in an image of the population of fungal cells; (b) determining values of the gradient in signal intensity at individual pixels in the image to identify edges of cells, where the gradient values are determined in at least two directions for the individual pixels; and (c) determining curvature values for at least some of the edges to thereby characterize those edges as elongated or not.

Still another aspect of the invention pertains to computer program products including machine-readable media on which are stored program instructions for implementing at least some portion of the methods described above. Any of the methods of this invention may be represented, in whole or in part, as program instructions that can be provided on such computer readable media. In addition, the invention pertains to various combinations of data and data structures generated and/or used as described herein.

These and other features and advantages of the present invention will be described in more detail below with reference to the associated figures.

### BRIEF DESCRIPTION OF THE DRAWINGS

FIG. 1 is a simplified cartoon depiction of the cell cycle of a fungal cell showing the cellular elongation that sometimes results from arrested progress at various stages in the cell cycle.

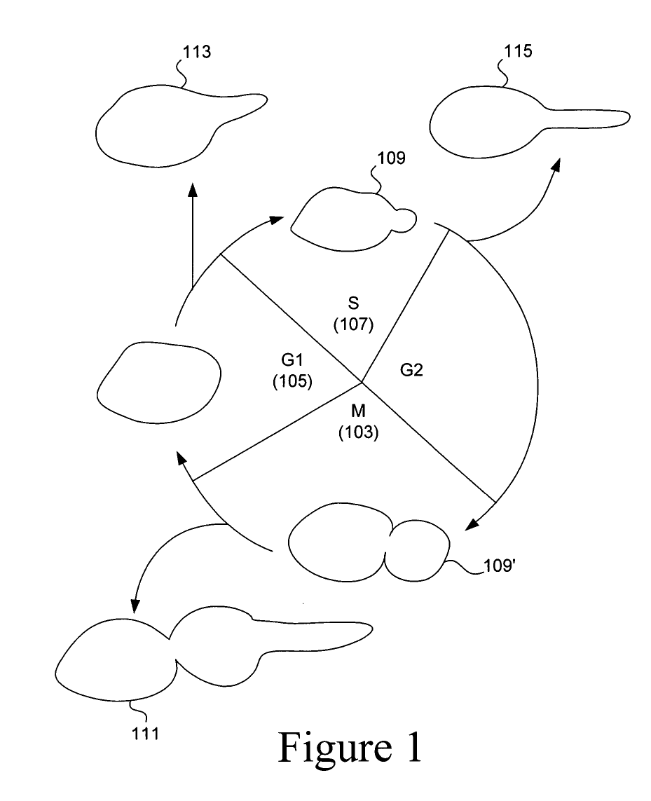

FIG. 2A is a cartoon drawing of normal fungal cells in culture. Relatively few of the elongation structures are observed.

FIG. 2B is a cartoon drawing of perturbed fungal cells in culture. Numerous examples of the elongation structures are observed.

FIG. 3 is a photo of Candida yeast cells illustrating four image processing issues of phase contrast microscopy.

FIG. 4 is a “processed” image showing series of “edges” derived from cell image in accordance with an embodiment of this invention.

FIG. 5 is a schematic depiction of general imaging system components that may be employed in accordance with this invention.

FIG. 6 is a process flow chart of one elongation characterization method of this invention.

FIG. 7 is a schematic image of a portion of an image showing a number of gradient values (at pixel positions) at different locations corresponding to (i) background, (ii) cell interiors, and (iii) cell edges.

FIG. 8 is a histogram of gradient value counts from an image. The histogram may be used in an adaptive thresholding technique known as triangular thresholding.

FIG. 9 is a schematic depiction of edges and how an automated method of this invention may characterize those edges as elongated or not.

FIG. 10 is a simplified block diagram of a computer system that may be used to implement various aspects of this invention, including characterizing populations of cells based on elongation.

### DETAILED DESCRIPTION OF A PREFERRED EMBODIMENT

Overview and Introduction

This invention characterizes biological cells or other biological features based on geometric elongation of the cells or features. This characterization gives researchers an additional tool for determining whether certain stimuli act by a particular mechanism of action, e.g., a mechanism that arrests progression through the cell cycle in fungal cells.

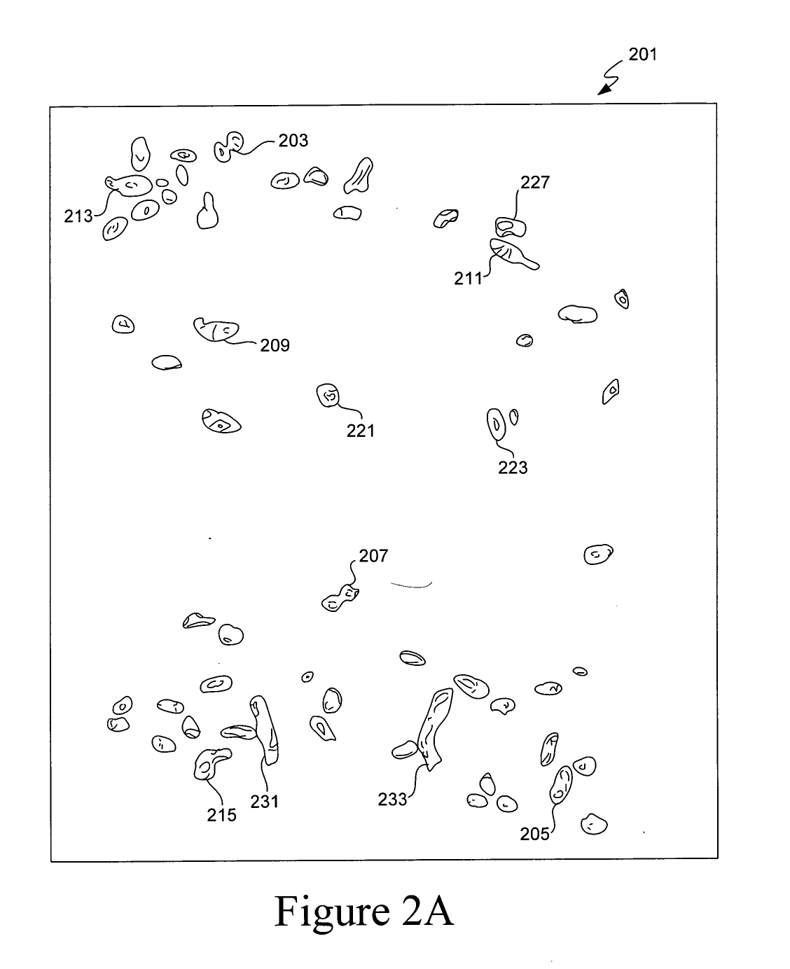

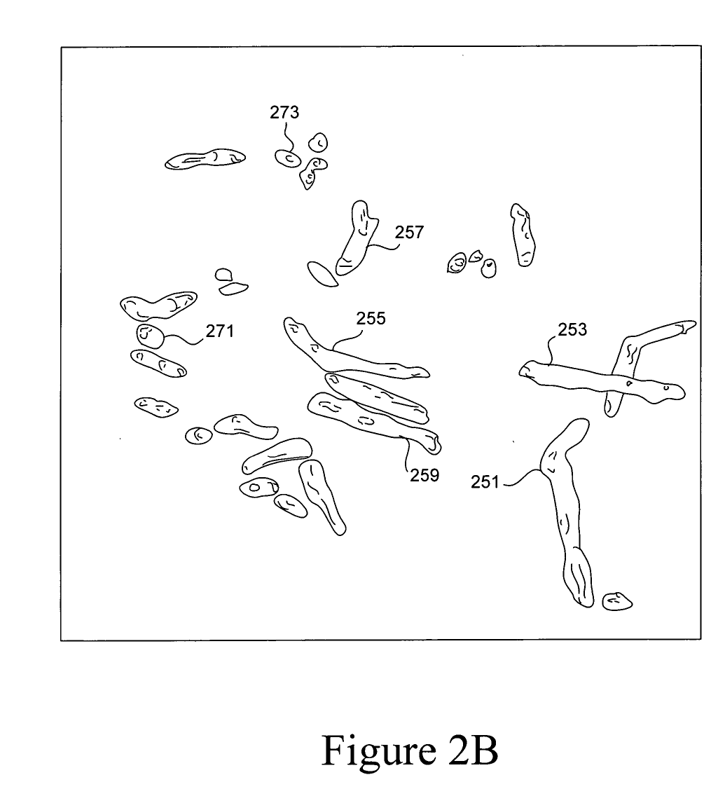

FIG. 2A presents a cartoon drawing of normal or untreated fungal cells 201 in culture. Many of the cells can be easily classified by the stage of the cell cycle where they reside. Cells 203, 205, and 207, for example, are in the M phase, while cells 209, 211, 213, and 215 are in the S or G2 phase, and cells 221, 223, 225, and 227 are in the G1 phase. A characteristic shape identifies each of these. In general, the fungal cells are relatively rounded without long linear features. As shown, there are a few cells with aberrant shapes (e.g., cells 231 and 233), but for the most part the cells in this figure do not include elongated structures.

FIG. 2B presents a cartoon drawing of fungal cells in culture having a relatively high percentage of “abnormal” elongated examples. This might result from the cells having been exposed to a stimulus that arrests progression at any one or more of the cell cycle phases. The cells so treated have developed certain elongation structures. See cells 251, 253, 255, 257, and 259, for example. As expected, some fungal cells do not exhibit the elongation. See cells 271 and 273.

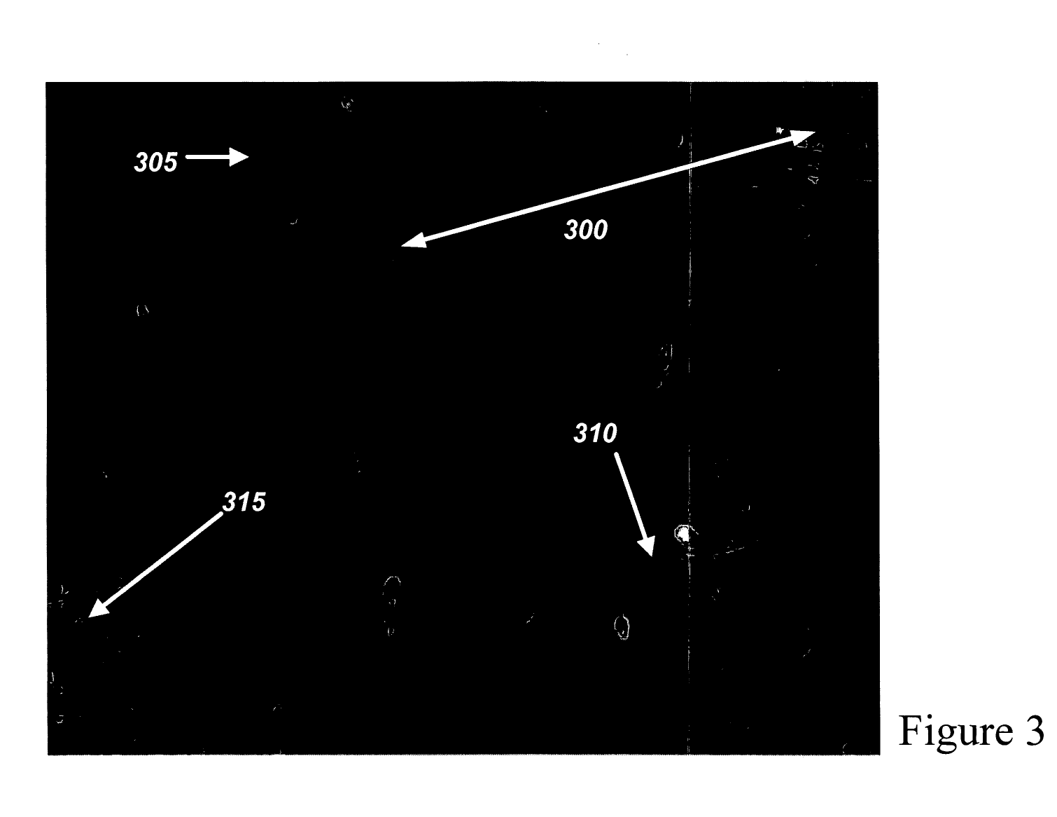

While this invention is not limited to any particular type microscopy, certain embodiments described herein employ phase contrast microscopy. Images generated from this form of microscopy present particular challenges, some of which are depicted in FIG. 3.

The phase contrast microscope is designed to take advantage of phase differences between objects in a specimen and in the surrounding medium. The light waves from the illumination source undergo phase shifts due to differences in optical path produced by differences in refractive index among components of the specimen. In the microscope, the optical path differences are amplified (often with a phase-plate to increase the phase difference to half of a wavelength) and the light waves are combined to produce regions of constructive and destructive interference. This improves the contrast at edges of the specimen. The phase contrast microscope and similar tools are particularly useful when dealing with transparent and colorless components in a cell. Dyeing the cells is an alternative but this may stop certain cellular processes and requires special pre-treatment of the specimen.

While phase contrast microscopy is one preferred means of producing high-contrast images for use in this invention, the invention can be applied to images generated by a number of other techniques. Generally, the chosen technique should produce an image having good contrast along the feature edges to be characterized. Light can interact with a specimen through a variety of mechanisms to generate the requisite contrast. Examples include absorption of light, reflectance, refraction, light scattering, diffraction, fluorescence, and color variations. Exemplary microscopy techniques include, in addition to phase contrast microscopy, bright field microscopy, Hoffman modulation contrast microscopy, differential interference contrast microscopy rely on variations in absorption or refractive index.

Some of the challenges surrounding image analysis in phase contrast images are depicted in FIG. 3, a phase contrast image of Candida cells. First, it can be difficult to distinguish cell features when cells are clumped closely together as shown in feature 315. Additionally, while using this technique makes transparent features of biological organisms more visible, it does not do so in a uniform fashion. One source of non-uniformity arises because phase differences between light rays vary over relatively small features of the image, e.g., cells. It is not uncommon that the light reaching the eyepiece may be more than one half of a wavelength out-of-phase. If the phase difference reaches a full wavelength (180°), a reversal of image contrast will occur, inverting light and dark patterns. Thus, some objects are dark and others are light. This is called signal reversal and is illustrated by feature 300. Another problem, shown in feature 305, is formation of “halos” around features of interest. These are artifacts and do not represent features of the cell culture. Further, cellular boundaries are often non-uniform, as illustrated in feature 310. In other words, on one side of the cell image, light changes from dark to light when moving from inside the cell to outside the cell. On the other side of the cell, the light changes from light to dark when moving from inside the cell to outside the cell.

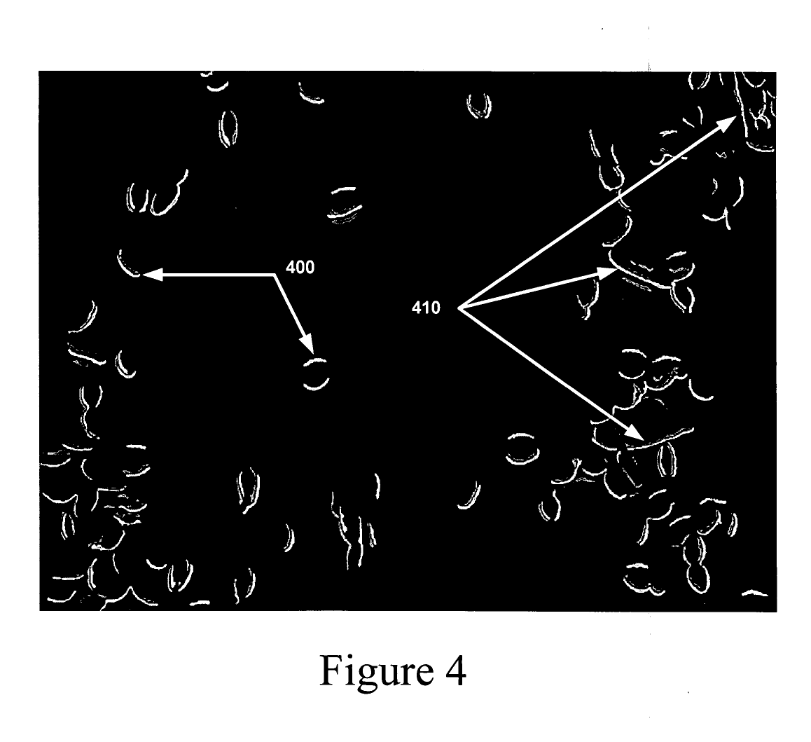

The present invention converts an initial image such as the phase contrast image of FIG. 3 to an image showing edges or boundaries of biological features. FIG. 4 is an “edge image” of this type that results from processing the FIG. 3 photograph in accordance with this invention. The edges of the cells in the photograph are now represented by thin curves that either define a circular region as shown in feature 400 or an elongated region as shown in feature 410. The algorithms of this invention take into account the spatial orientation (e.g., vertical or horizontal) of each of the edges and characterize each edge as either sufficiently circular (normal) or elongated (abnormal).

Definitions

Some of the terms used herein are not commonly used in the art. Others may have multiple meanings in the art. Therefore, the following definitions are provided as an aid to understanding the description that follows. The invention as set forth in the claims should not necessarily be limited by these definitions.

The term “stimulus” refers to something that may influence the biological condition of a cell. Often the term will be synonymous with “agent,” “treatment” or “manipulation.” Stimuli may be materials, radiation (including all manner of electromagnetic and particle radiation), forces (including mechanical (e.g., gravitational), electrical, magnetic, and nuclear), fields, thermal energy, and the like. General examples of materials that may be used as stimuli include organic and inorganic chemical compounds, biological materials such as nucleic acids, carbohydrates, proteins and peptides, lipids, various infectious agents, mixtures of the foregoing, and the like. Other general examples of stimuli include non-ambient temperature, non-ambient pressure, acoustic energy, electromagnetic radiation of all frequencies, the lack of a particular material (e.g., the lack of oxygen as in ischemia), temporal factors, treatments that reduce or eliminate expression of one or more genes, etc.

Specific examples of biological stimuli include exposure to hormones, growth factors, antibodies, or extra-cellular matrix components. Or exposure to agents such as infective materials such as viruses that may be naturally occurring viruses or viruses engineered to express exogenous genes at various levels. Biological stimuli could also include delivery of antisense polynucleotides by means such as gene transfection. Stimuli also could include exposure of cells to conditions that promote cell fusion. Specific physical stimuli could include exposing cells to shear stress under different rates of fluid flow, exposure of cells to different temperatures, exposure of cells to vacuum or positive pressure, or exposure of cells to sonication. Another stimulus includes applying centrifugal force. Still other specific stimuli include changes in gravitational force, including sub-gravitation, application of a constant or pulsed electrical current. Yet other stimuli include incubation in the presence of small (often organic) molecules that may affect cells. Still other stimuli include irradiation, photo bleaching, which in some embodiments may include prior addition of a substance that would specifically mark areas to be photobleached by subsequent light exposure. In addition, these types of stimuli may be varied as to time of exposure, or cells could be subjected to multiple stimuli in various combinations and orders of addition. Of course, the type of manipulation used depends upon the application.

The term “phenotype” generally refers to the total appearance of an organism or cell from an organism. In the context of this invention, cellular phenotypes may be represented in terms of its “elongation” and optionally other parameters, which may be stored in and manipulated by processing systems (e.g., computers). A given cell's phenotype is a function of its genetic constitution and environment. Often a particular phenotype can be correlated or associated with a particular biological condition or mechanism of action resulting from exposure to a stimulus. Cells undergoing a change in biological condition will frequently undergo a corresponding change in phenotype. Thus, cellular phenotypic data and characterizations may be exploited to deduce mechanisms of action of a stimulus and other aspects of cellular responses to various stimuli.

The term “path” or “response curve” refers to the characterization of a stimulus at various levels and/or at different times after application of a stimulus. For example, the path may characterize the effect of a chemical applied at various concentrations or the effect of electromagnetic radiation provided to cells at various levels of intensity or the effect of depriving a cell of various levels of a nutrient. In another example, the path characterizes the effect of a stimulus at various times after the stimulus was initially applied. Mathematically, the path is made up of multiple points, each at a different level of the stimulus and/or time point. In accordance with this invention, each of these points is preferably a collection of parameters or characterizations describing some aspect of a cell or collection of cells. Typically, at least some of these parameters and/or characterizations are derived from images of the cells. In this regard, they represent signatures of the cells. In the sense that each point in the path may contain more than one piece of information about a cell, the points may be viewed as arrays, vectors, matrices, etc. To the extent that the path connects points containing phenotypic information (separate quantitative phenotypes), the path itself may be viewed as a phenotype that is independent of a “stimulus level.”

Note that in the context of this document, there will be numerous references to yeast and fungal cells. Generally, the invention pertains to any fungal cell. So when the document refers to “yeast,” fungal cells generally are contemplated. In many contexts the invention, including the underlying algorithms and assays, applies more generally to all or many cell types, even that from part of a tissue culture.

Generating the Image

After the relevant strains or cell lines have been selected, each individual strain or cell line is prepared for imaging. Preferably, this process is performed in a high-throughput automated manner, possibly with the aid of a robot. The production of the images may include cell plating, cell treatment (e.g., compound dilution and compound addition) and imaging.

Initially, the cells of the selected strain are grown in an appropriate medium (e.g., YPD for fungal cells, Adams et al. 1997, Methods in Yeast Genetics, Cold Spring Harbor Laboratory Press, incorporated herein by reference for all purposes). In one embodiment, yeast cells are grown at 30 degrees Centigrade. After the cells have been grown for a defined period (e.g., 3 population doublings), they are treated and imaged.

Note that certain cells such as yeast cells have a propensity to aggregate or “clump.” Clumped cells are difficult to analyze with image analysis software because edges may be difficult to distinguish. Therefore, the process may include an operation that reduces the likelihood that cells will clump, e.g., sonication. In an alternative or complementary approach, image analysis may also include some preprocessing or filtering to remove “clumped” cells from consideration. Clumped cells are easily identifiable by their relatively large size and/or atypical shapes. Software that recognizes such clumps can be used to separate the clumped and unclumped yeast cells in an image.

The cells are typically treated with a selected agent or stimulus as described above. In many important applications, the stimulus is a chemical agent. Typically, a chemical agent is delivered in a solution and/or with other compounds or treatments, and at varying dose levels. The cells may also be exposed to a biological treatment, such as a virus, protein or by having the cells' DNA modified by any other means by which biological effects may be induced in the cells.

Experimental protocols for investigating the effect of a treatment will be apparent to a person of skill in the art and can include variations in the dose level, incubation time, cell type, cell line and other parameters, which are typically varied as part of an experimental protocol. In a typical case, an experiment on the effect of a treatment is carried out by combining sets of assay plates. An assay plate is usually a collection of wells arranged in an array with each well holding at least one cell or a related group or population of cells which have been exposed to a treatment or which provides a control group, population or sample. In other embodiments, multiwell plates are not used and single sample holders can be used. Because yeast cells do not adhere well to plastic substrates, the plates on which they are to be imaged may be coated with an adherent material such as polylysine.

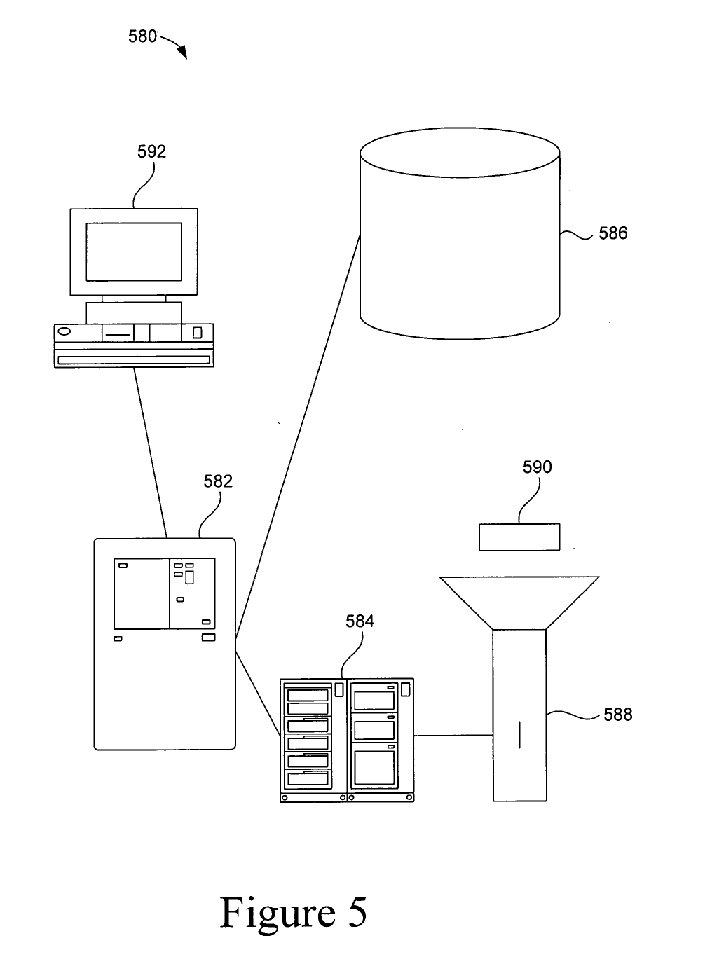

FIG. 5 shows a schematic block diagram of an image capture and image processing system 580 which can be used to capture and process the images of cells or cell parts and store the cellular features. This diagram is merely an example and should not be construed to limit the scope of the invention. The system 580 includes a variety of elements such as a computing device 582, which is coupled to an image processor 584 and is coupled to a database 586. The image processor receives information from an image-capturing device 588, which includes an optical device for magnifying images of cells, such as a microscope. The image processor and image-capturing device can collectively be referred to as the imaging system herein. The image-capturing device obtains information from a plate 590, which includes a plurality of wells providing sites for groups of cells. These cells can be cells that are living, fixed, cell fractions, cells in a tissue, and the like. The computing device 582 retrieves the information, which has been digitized, from the image-processing device and stores such information into the database 586. A user interface device 592, which can be a personal computer, a work station, a network computer, a personal digital assistant, or the like, is coupled to the computing device.

As described above, many types of microscopy may be employed in the imaging process. In many preferred embodiments, the image is produced by a technique that captures variations in at least one of absorbance and refractive index. Again, exemplary types of microscopy include at least one of phase contrast microscopy, Hoffman modulation contrast microscopy, differential interference contrast microscopy, and bright field microscopy. Preferably, live cells are imaged and the imaging technique does not appreciably perturb the cells. Thus, many imaging techniques used with this invention will not involve staining or fixing the cells.

Given the relatively small size of yeast cells, they are preferably imaged at a magnification of between about 200× and 400×, employing for example 20× and 40× objectives, respectively, in combination with a 10× photo ocular. In addition, the imaging system is preferably designed to auto-focus on cells at that magnification level.

The resulting image comprises image data showing signal intensity or type (e.g., color) versus position in the image. The signal variation reflects phenotypic features of the population of cells being imaged. Discrete positions in an image are usually defined by pixels.

Example Algorithm for Characterizing Elongation

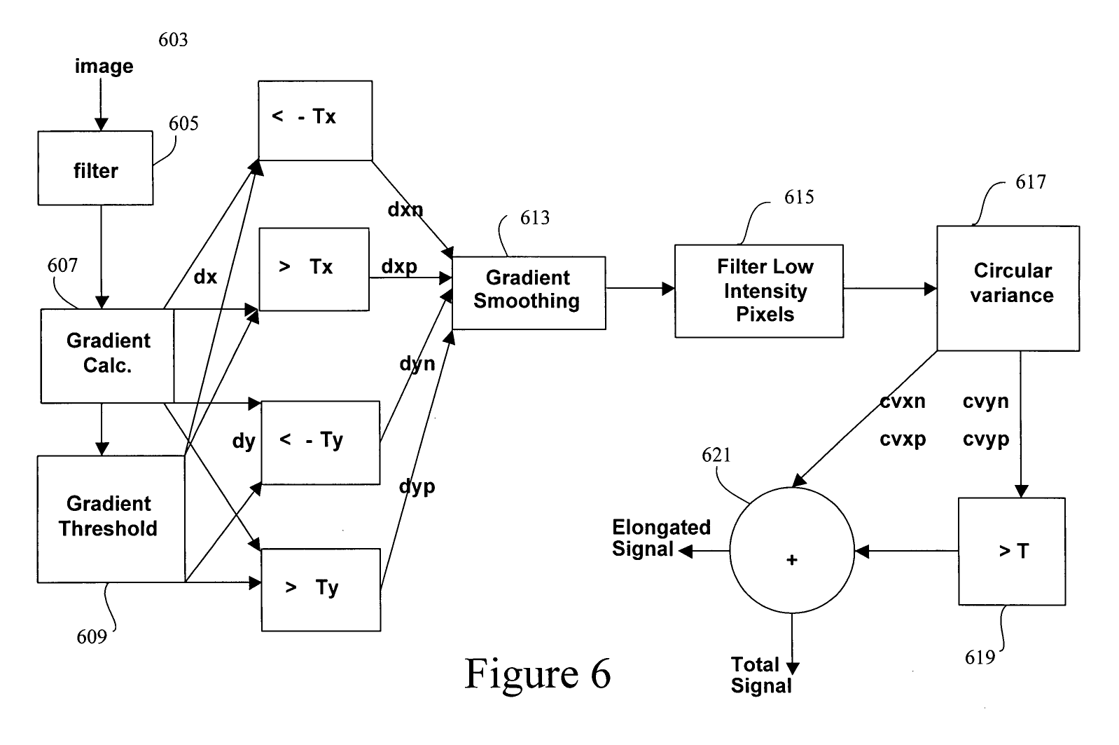

FIG. 6 presents, by way of a flowchart, one example of a generalized process for implementing the present invention. As depicted, the process begins with receipt of an image 603 depicting a biological system of interest. The image may be produced by any of the various techniques described above or others. Initially in the depicted embodiment, the image is “smoothed” or otherwise filtered to reduce very abrupt pixel-by-pixel intensity variations. See block 605. The image can be corrected to remove any artifacts introduced by the image capture system. In a specific embodiment, a correction algorithm is applied to correct for changing light conditions, positions of wells, etc. In one example, a noise reduction technique such as median filtering is employed. A correction for spatial differences in intensity also may be employed. Applying the correction values to the image adjusts for optical system non-linearities, mis-positioning of wells during imaging, etc.

Next, at block 607, the process continues by analyzing the image on a location-by-location or pixel-by-pixel basis in which, at each location or pixel, a local gradient value is determined. This can be accomplished in various ways. One method is to simply consider two adjacent pixels and calculate a change in intensity in the direction connecting the two pixels. In another approach, the process calculates two separate gradients at each pixel or location: one in the horizontal direction and another in the vertical direction. Each of these is then saved. In a more general approach, any number of different directions can be utilized in calculating the gradients. Further, the gradient need not be limited to variations in intensity between immediately adjacent pixels; rather it can span variations across multiple pixels in various directions.

Large gradient values suggest a possible edge region in the image. To this end, the gradient values are compared against a gradient threshold. Such threshold may be fixed (i.e., independent of any particular distribution of gradient values obtained from the image itself) or adaptive (i.e., derived from one or more actual images of the biological system or systems under investigation). Assuming that an adaptive threshold technique is employed (i.e., a threshold is calculated from the image itself,) the process of FIG. 6 depicts an operation for calculating such gradient threshold. See block 609. As described below, a histogram technique for calculating an adaptive threshold can be used. In the specific embodiment depicted, separate gradient thresholds are calculated for horizontal and vertical direction gradients.

Typically one wants to capture gradient variations that are large in both positive and negative directions. This is because some images transition from light to dark in a particular direction while others transition from dark to light in that direction. Therefore, it may be convenient to use an absolute value of gradient or simply compare the gradient to both a positive and a negative value of the threshold. This is what is depicted in FIG. 6 to the right of the gradient and threshold blocks. As shown, there are four possibilities: (i) the gradient value at a given pixel in the x direction is greater than a positive value of the horizontal threshold (Tx), (ii) the gradient value in the x direction is more negative than a negative value of the horizontal threshold (−Tx), (iii) the gradient value in the y direction is greater than a positive value of the value of the vertical threshold (Ty), and (iv) the gradient value in the y direction is more negative than a negative value of the vertical threshold (−Ty). In this embodiment, a separate gradient value is calculated for each of the directions under consideration.

At the end of this process, those positive and negative gradient values that are greater than the associated threshold values are selected. So, in a sense, a revised image of large gradient values is generated. Many of these large gradient values will represent portions of the edges of the biological systems in question. Generally, the process views contiguous groups of high-gradient pixels as potential edges.

The “edge image” generated from the threshold comparisons may be separately filtered to remove various artifacts. In FIG. 6, the process includes optional filtering steps 613 and 615. Gradient smoothing operation 613 performs a similar noise-reduction function as performed by filtering the raw image at block 605 (described above). Filtering the low intensity pixels at 615 is intended to remove large gradient values that result from halos and other similar artifacts in the image. This is particularly valuable in some types of phase contrast images. One approach to filtering uses a heuristic threshold (e.g., a given quantile, say 80 percentile) based on the assumption that the real edge points are very bright and the intensities are above 80 percent of all pixels.

With the edge image appropriately filtered if necessary, the edges can now be analyzed to characterize their shape. See block 617. As indicated above, one shape criterion of particular relevance in the context of this invention is “elongation.” Normally, one would expect to find relatively circular edges in many biological systems (e.g., healthy fungal cells). As explained in more detail below, the circular variance of an edge is one useful metric for characterizing the elongation of a biological system.

In the depicted embodiment, the process calculates the circular variance separately for each of the four types of identified edges: positive x direction, negative x direction, positive y direction, and negative y direction. The circular variance of each edge is then compared against a threshold as depicted in block 619 to classify individual edges as elongated or not. In other words, the output is True or False. In another embodiment, each edge is given a separate numerical value representing the degree of elongation or other shape characterization.

In the depicted embodiment, the total number of pixels associated with “elongated edges” in the image is compared to the total number of edge pixels. See comparison block 621. This generates both a total signal showing all edges and an “elongated edges” signal showing only those edges that are deemed to be elongated by the shape characterization operation. The degree of elongation in the overall image may be characterized as a ratio of the number of pixels in the elongated signal to the total number of edge pixels.

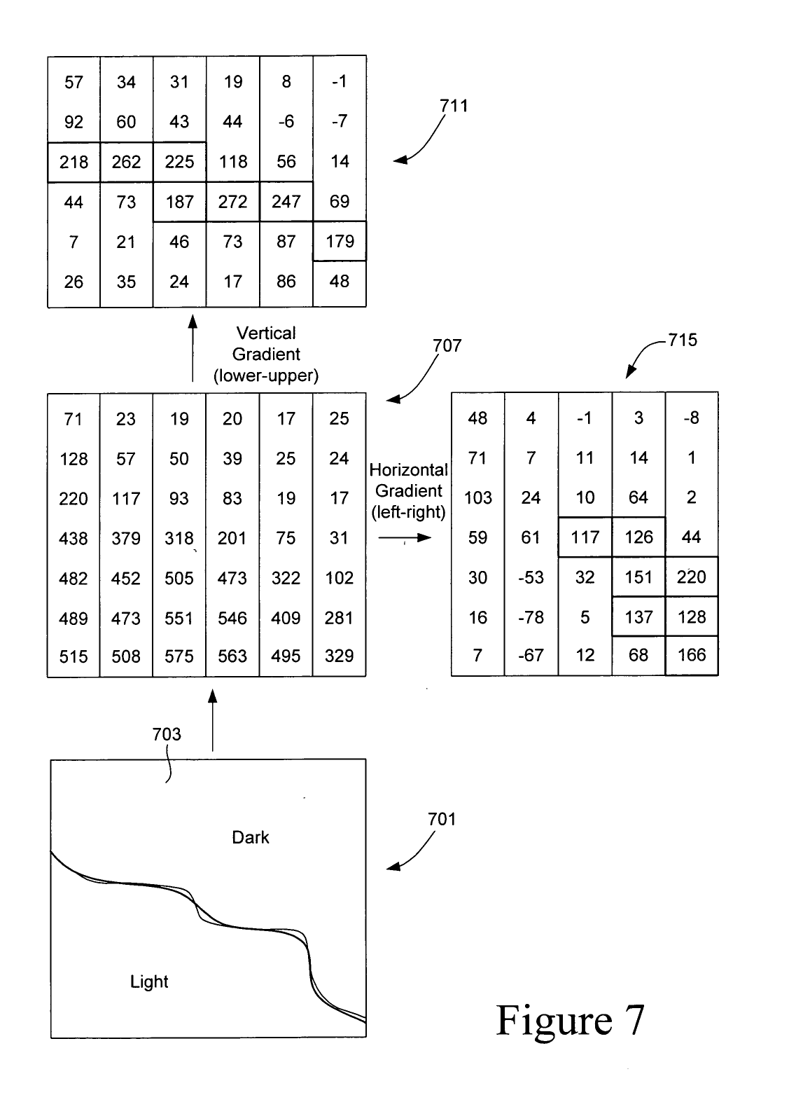

FIG. 7 illustrates one approach to identifying large gradient values within an image of a biological system. As shown, a portion 701 of an image includes a generally dark region 703 and a generally bright region 705, separated by a boundary. As shown in the depiction of portion 701, the boundary between the dark and bright regions has vertical and horizontal components. The boundary may represent, for example, an edge of a cell in an image. Note that each location of the image has an associated gradient value, which can be derived from local values of image parameters.

Image portion 701 may be represented as a collection of pixels 707, each representing a location in the image and having an associated value of intensity of other optical property captured in the image. In FIG. 7, each pixel is represented by a block with a numerical value of intensity; with larger values representing brighter pixels. These intensity values can be converted to gradient values. In the simple case, vertical gradient values are obtained by selecting two vertically adjacent pixels and subtracting the upper pixel value from the lower. Each pixel is considered in this manner. After considering every pixel in the image, the process has generated a vertical gradient image 711. A similar method generates a horizontal gradient image 715. In the horizontal case, two horizontally adjacent pixels are chosen and the intensity value of the right one is subtracted from the left one.

In addition to the simple pixel-to-pixel horizontal and vertical gradient calculations, algorithms of this invention may employ more complex gradient calculations. For example, various techniques can calculate a gradient over non-adjacent pixels. Other techniques consider three or more pixels. Further, the calculation may include simultaneous consideration of both horizontally and vertically separated pixels. Such calculation may be represented by a “window” or matrix; e.g. a matrix having 3 rows and 2 columns with multiplier values of 0, −1, and 1. Note that calculations are not constrained to the horizontal and vertical directions. They can also be conducted in a diagonal direction, for example.

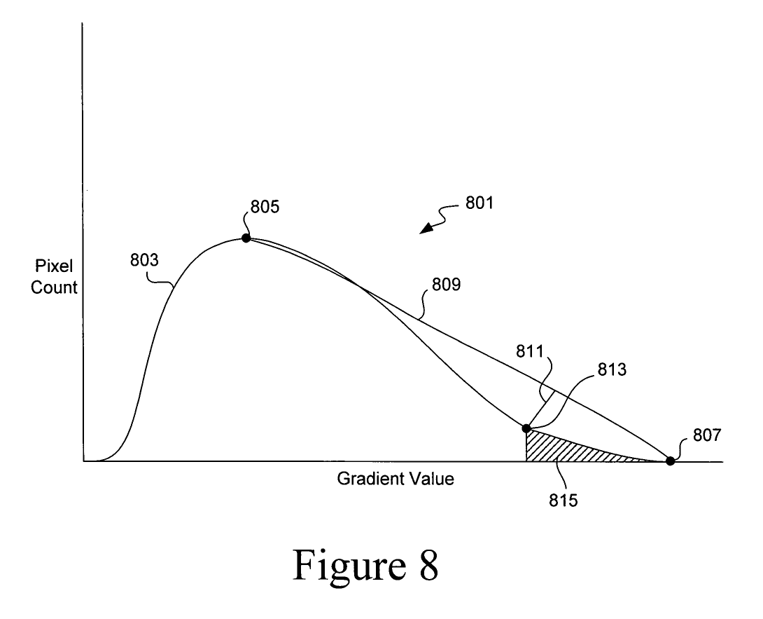

As shown in the vertical and horizontal gradient images 711 and 715 of FIG. 7, pixels having gradient values of greater than a particular threshold value are “selected” (as indicated by highlighted borders). In this example, the threshold value for the vertical gradient is 150, while the threshold value for horizontal gradient is 110. Gradient values greater than these thresholds are presumed to be associated with edges. As indicated, the threshold values may be obtained by various methods such as that depicted in FIG. 8.

As mentioned, a fixed or adaptive threshold may be employed to identify which gradient values in an image to represent potential edges of a biological feature. Fixed threshold features are determined empirically or heuristically, independently of a specific analysis of the image under consideration. A fixed threshold value may be appropriate in cases where particular gradient values consistently correlate with edges of the biological features in a number of different images. An adaptive threshold is needed where this is not the case. In contrast to a fixed threshold, which is set independently of any information gained from images under consideration, an adaptive threshold is determined from the image data itself. Various techniques can be used for this purpose. In many cases, the adaptive threshold is calculated using a histogram of pixel property values for the image under consideration. A separate threshold may be calculated for each direction of gradient of consideration, as in the example of FIG. 6 where separate gradient thresholds were calculated for the horizontal and vertical directions.

One approach to calculating an adaptive threshold is known as a triangular threshold technique. This approach can be understood visually by referring to FIG. 8. As shown there, a histogram 801 provides the pixel count versus gradient value. Within a single image, each gradient value occurs never, once, or many times. The number of pixels having a particular gradient value is the y component of the coordinates of a curve 803. In the triangular threshold technique, an apex of the histogram and an end of the tail of the histogram 807 are identified. A line 809 connects these two points. Then, a line 811 is identified. This line is perpendicular to line 809 and represents the greatest perpendicular distance from line 809 to the histogram curve 803. As shown, perpendicular line 811 intersects the histogram plot 803 at a point 813. The gradient value (x value) of point 813 represents the adaptive threshold value. All gradient values greater than 813 are deemed to be potential edge values in the underlying image. Thus, considering histogram 801, all pixels having gradient values in region 815 are deemed to be potential edge positions on the underlying image.

As suggested above, it may be necessary to perform an adaptive threshold analysis multiple times on a single image, once for each gradient direction under consideration. Thus, for example, a triangular threshold operation may need to be performed once for gradients calculated in the x direction and once for gradients calculated in the y direction. The gradient values in histogram 801 can be either absolute values or original values. In either case, the separate adaptive threshold could be calculated for both the positive and negative gradient values with the triangular threshold method.

After the vertical and horizontal threshold values, Tx and Ty, are derived (or any other appropriate threshold values depending on the types of gradient calculated), the positive and negative value edge images are derived. In the simple case of horizontal and vertical gradients, four separate edge images are produced: (1) dark to bright horizontally (dp/dx <−Tx), (2) bright to dark horizontally (dp/dx>Tx), (3) dark to bright vertically (dp/dy <−Ty), and (4) bright to dark vertically (dp/dy>Ty). A superposition? of all four edge images will appear as, for example, FIG. 4 described above. Typically, adjacent pixels or pixels separated by less than a defined maximum separation distance are grouped and treated as an edge. In some embodiments, an edge requires at least a defined minimum number of pixels. In other words, two adjacent pixels would not, of themselves, be deemed an edge.

Note that in the method of FIG. 6, the edges are identified without actually identifying individual cells or positions of cells. Only edges need be detected.

As indicated in the flow chart of FIG. 6, methods of this invention typically characterize the shapes of the various edges identified in an image. While this characterization may be accomplished in many different ways, a “shape descriptor” is conveniently employed for this purpose. Shape descriptors that characterize the degree of curvature possessed by an edge are particularly preferred. Examples of shape descriptors that can be used for this purpose include circular variance, curvature augmented with the major axis, an axis ratio which is the ratio of the major axis over the minor axis of the ellipsoid best fitted to the shape, etc. Circular variance will be described as an example of a shape descriptor that can be used in accordance with this invention.

As the name suggests, circular variance represents the deviation of a particular shape or edge from a true circle. The goal is to distinguish elongated shapes from generally circular shapes. Shapes with a greater degree of elongation will have a larger value of circular variance.

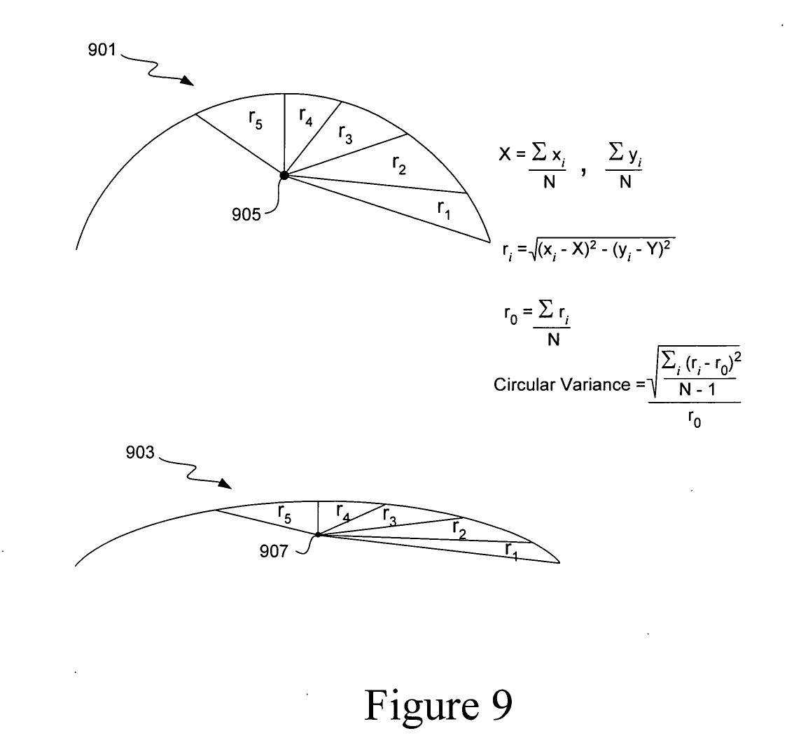

The concept of circular variance is illustrated in FIG. 9. Initially, the method calculates a centroid for an edge under consideration in the image. The centroid (X, Y) represents the coordinate of the mean value of X and the mean value of Y in the edge under consideration. In a relatively circular edge 901 depicted in FIG. 9, the centroid is given by a point 905. In a relatively elongated edge 903, the centroid is represented by a point 907.

Once the centroid of an edge is identified, the radii between the centroid and each edge point are calculated. As shown in FIG. 9, these are indicated by the r_{i (r}₁, r₂, r₃ . . . ). From these radii, a mean radius value r₀ is calculated for the edge under consideration. With this mean value and the individual radii, the circular variance can be calculated as illustrated in FIG. 9. Note that the parameter “N” represents the total number of points considered in the edge. Edges with a greater range in the value of their individual radii will give greater values of circular variance and thereby be characterized as elongated in accordance with this invention.

Individual curves are characterized as elongated or not based on whether the value of their shape descriptor exceeds a threshold for elongation (sometimes referred to herein as an “elongation threshold”). Such thresholds can be calculated as fixed thresholds or adaptive thresholds. In one example that involves characterizing images of fungal cells, circular variance is used as a shape descriptor and a value of 5 has been determined empirically to work well threshold value. In other words, edges having a circular variance of 5 or higher are deemed to be elongated. Other thresholds can be derived adaptively from a histogram of circular variance values in an image under consideration. Of course, the triangular method is one way to calculate such adaptive threshold value.

The overall “quantity” of elongation in any given biological system under consideration can be computed from the shape characterization of the individual edges in an image. Generally, a method will specify a ratio or fraction of the number of pixels in elongated curves to the total number of pixels in all curves of the image. This is a pixel-based characterization of elongation. Alternatively, the method may characterize elongation in terms of a ratio or fraction of the number of curves that are elongated to the total number of curves in the image.

Note that above techniques first determine whether individual curves are elongated or not, and then use those classifications to characterize the overall image. All curves or pixels deemed to be elongated are weighted equally in the overall characterization of the image. In other words, curves that are highly elongated provide the same contribution as curves that are moderately elongated. In an alternative approach, the shape descriptor value can be used as such to provide a weighted contribution to elongation within the image.

Applications

There are many applications of the present invention. Some have been mentioned already. In many instances, the degree of elongation in a cell or population of cells is strong indicator of a particular mechanism of action for an applied stimulus. One mechanism of action for which elongation is strong indicator is interruption of the cell cycle in fungal cells, including yeast cells. Compounds acting by this mechanism are candidate drugs for treating fungal infections. Thus, an important application of the present invention is as a screen in finding new anti-fungal agents. Another application is in monitoring an existing series of compounds for “on target” effects; e.g., effects that are manifested by increased or decreased cellular elongation.

In certain embodiments of the invention, elongation assays are applied to populations of yeast cells. Yeasts (including Saccharomyces and Candida) are a subset of fungi. Importantly, yeasts and other fungi can manifest as human pathogens, often resulting in debilitating disease states or death. In some cases, the yeast or other organism under consideration can be genetically modified.

Generally, phenotypic information from fungal cells can be obtained from genetic manipulation and/or environmental stress. Examples of such stress includes high temperatures (e.g., between about 34 and 42 degrees Centigrade), low temperature (e.g., between about 10 and 20 degrees Centigrade), high salt concentration (e.g., between about 0.5M and 1M ionic species in the media), and the presence of specific chemical agents (e.g., candidate drugs as mentioned). Examples of other stress inducing conditions include using minimal quantities of media and nitrogen starvation. Examples of chemical agents include toxins, suspected toxins, drugs, and drug candidates. From a more specific biochemical perspective, examples of chemical agents include pheromones (e.g., α-factor), actin depolymerization agents, and microtubule depolymerization agents (e.g., benomyl). Other examples include antifungal drugs such as various azoles, 5-fluorocytosine, griseofulvin, terbinafine, and amphotericin B. Each of these different stresses produces a separate phenotypic fingerprint generated by imaging the associated cells and quantifying features, including cellular elongation, in those images.

Another application of the present invention is in characterizing the transition of fungal cells to a hyphal phase, where they develop elongated filamentous threads (hypha) that make up fungal mycelia. The image analysis methods of this invention can be used to characterize cells on the basis of their transition to the hyphal state. This analysis can be used to research fundamental mechanisms of the transition and/or evaluate a treatment intended to affect transition to the hyphal state. Transition between hyphal and non-hyphal states is required for virulence of many fungi, and compounds that modulate this transition are candidate drugs for treating fungal infections.

Outside the context of fungal assays, there are other mechanisms of action that might be manifested by elongation in a cell population. These include elongation of axons during nerve cell growth and differentiation, and defects in bacterial cell division which lead to formation of chains of connected cells. Compounds that modulate this transition bacterial cell division are candidate drugs for treating some bacterial infections.

Elongation characterizations derived from methods of this invention can be used with other image-derived descriptors to characterize a phenotype. A selected collection of data and characterizations that represent a phenotype of a given cell or group of cells is sometimes referred to as a “signature” or “quantitative cellular phenotype.” This collection is also sometimes referred to as a phenotypic fingerprint or just “fingerprint.” The multiple cellular attributes or features of the signature can be collectively stored and/or indexed, numerically or otherwise. For convenience, phenotypic fingerprints can be treated as data structures by database and algorithmic software. Mathematically, the fingerprints may be viewed as vectors, each comprised of several scalar values. For certain phenotypic comparisons, these scalar values may be weighted differently.

Functionally, the attributes making up the fingerprint are typically quantified in the context of specific cellular components or markers. In addition, to degree of elongation, other measured attributes useful for characterizing an associated phenotype include morphological descriptors (e.g., size, shape, and/or location of cells or sub-cellular cellular organelles) and composition (e.g., concentration distribution of particular biomolecules within the cells or organelles). Other attributes include changes in a migration pattern, a growth rate, hypha formation, an extracellular matrix deposition, and cell count.

In one example, a given fungal cell population may have a first phenotypic fingerprint for normal growth conditions (e.g., rich media at 30 degrees Centigrade as mentioned above), a second phenotypic fingerprint for growth at elevated temperatures, a third phenotypic fingerprint for growth in highly saline conditions, a fourth phenotypic fingerprint for exposure to a particular drug, etc. As indicated, the fingerprints are comprised of various quantitative and/or qualitative values (e.g., the cell is in cell cycle phase “n” and has an actin polarization of “x” microns) and possibly some yes/no characterizations (e.g., the cell is budding).

The elongation data for a particular experiment, used alone or in more complex phenotypic fingerprints, can serve as an individual point on a response curve. A phenotypic response to stimulus may be characterized by exposing cells to a stimulus of interest at various levels (e.g., concentrations of a compound). In each level within this range, the phenotypic descriptors of interest (including elongation data) are measured to generate a phenotypic fingerprint associated with the level of stimulus. Typically, the response curve includes a “zero point” represented by the phenotype measured in a control system, where the stimulus is absent. Another point is the phenotypic characterization of the cells when exposed to a low level of stimulus (e.g., a relatively low concentration of a chemical compound). Another point is the phenotypic characterization of the cells when exposed to a somewhat higher concentration of the chemical compound, and so on.

Many important applications investigate the dose response of a stimulus on a biological system. The potency and certain other features of the stimulus under investigation can be assessed by comparing a control phenotypic characterization (including degree of elongation) to a low dose phenotypic response of a biological system. Cells having only low or moderate elongation response suggest a low potency stimulus. The trajectory or shape of the dose response curve can also provide meaningful information to characterize a mechanism of action or other biological response. Stimuli with “similar” trajectories may be viewed as likely acting by a similar mechanisms of action. Methods for generating and analyzing dose response curves are described in U.S. patent application Ser. No. 09/789,595, filed Feb. 20, 2001 and in U.S. patent application Ser. No. 10/623,485, filed Jul. 18, 2003.

It will often be convenient to store the elongation date, sometimes in conjunction with other phenotypic information, in a database or “knowledge base.” The phenotype information may be organized within such database in a variety of ways. In one embodiment, each cell image presents a unique record. In another embodiment, each unique combination of cell type and applied stimulus is uniquely identified. The elongation characterization or other quantitative representation of a phenotype is stored in an appropriate data record or at least pointed to by an indexed record. The data records may also specify a separation “distance” of the phenotype at issue from other phenotypes. The distance may have a numeric value (e.g., an average, a weighted average, a Euclidean distance, etc.). Still further, the database records may identify how the cells under consideration are grouped or clustered.

Beyond the above examples in which the invention characterizes the overall cell shape, the invention can be easily extended to characterize the edges of any biological feature in an image. For example, rather than merely characterizing the gross cell shape, the invention can be used to characterize the shape of nuclei or other organelles or sub-cellular features within a cell. The invention can also characterize the elongation of super-cellular features such as lumens in a tissue sample or cellular aggregations arising in some cultures. Of course, the microscopy and imaging conditions will be set to emphasize the contrast in the features of interest.

Further, many examples presented herein identify exposure to chemical compounds as the stimuli of interest. The invention can be used to characterize many different stimuli, not just exposure to chemical compounds. As mentioned, relevant stimuli include exposure to biological agents, exposure to various fields, forces, and radiation, deprivation of agents important for normal cell growth and functioning, etc.

Still further, the invention can be used with many different types of images. While many embodiments employ images in which live cells are used, some other embodiments employ images of cells exposed dyes. In such embodiments, the cells are marked to emphasize certain features in an image. Selection of appropriate markers requires balancing certain considerations. First, a marker should be chosen to highlight an interesting, informative feature of the cells. For example, a marker may highlight a cell wall or cell membrane, a sub-cellular organelle, or a cellular biomolecule. Second, a marker should not significantly interfere with the cellular phenotype. In preferred embodiments, for example, yeast markers should be able to penetrate the cell wall without damaging it. For this reason, it is generally preferred that non-immunological markers be used to mark yeast cell features. Antibodies and antibody components are too large to pass through the yeast cell wall without having first modified the cell wall. Another consideration in selecting markers is the ease with which they may be applied to yeast cells (preferably fixed yeast cells in suspension or living yeast cells in suspension).

Software/Hardware

Certain embodiments of the present invention employ processes acting under control of instructions and/or data stored in or transferred through one or more computer systems. Embodiments of the present invention also relate to an apparatus for performing these operations. This apparatus may be specially designed and/or constructed for the required purposes, or it may be a general-purpose computer selectively configured by one or more computer programs and/or data structures stored in or otherwise made available to the computer. The processes presented herein are not inherently related to any particular computer or other apparatus. In particular, various general-purpose machines may be used with programs written in accordance with the teachings herein, or it may be more convenient to construct a more specialized apparatus to perform the method steps. A particular structure for a variety of these machines is shown and described below.

In addition, embodiments of the present invention relate to computer readable media or computer program products that include program instructions and/or data (including data structures) for performing various computer-implemented operations associated with analyzing images of cells or other biological features, as well as classifying stimuli on the basis of how well they promote elongation of biological features (e.g., elongation of fungal cells). Examples of computer-readable media include, but are not limited to, magnetic media such as hard disks, floppy disks, and magnetic tape; optical media such as CD-ROM disks; magneto-optical media; semiconductor memory devices, and hardware devices that are specially configured to store and perform program instructions, such as read-only memory devices (ROM) and random access memory (RAM). The data and program instructions of this invention may also be embodied on a carrier wave or other transport medium (including electronic or optically conductive pathways).

Examples of program instructions include low-level code, such as that produced by a compiler, as well as higher-level code that may be executed by the computer using an interpreter. Further, the program instructions may be machine code, source code and/or any other code that directly or indirectly controls operation of a computing machine in accordance with this invention. The code may specify input, output, calculations, conditionals, branches, iterative loops, etc.

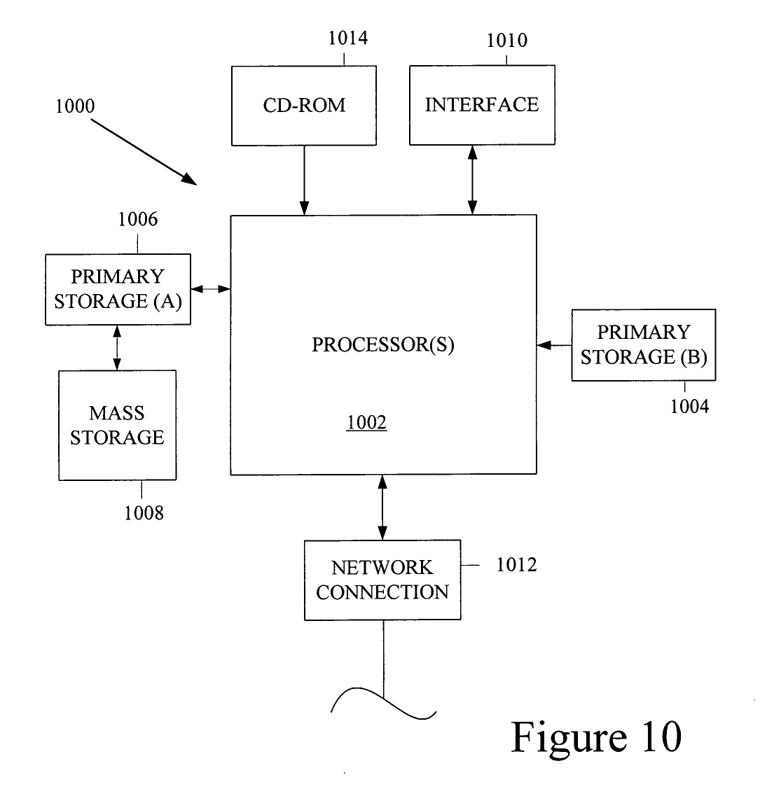

FIG. 10 illustrates, in simple block format, a typical computer system that, when appropriately configured or designed, can serve as a computational apparatus of this invention. The computer system 1000 includes any number of processors 1002 (also referred to as central processing units, or CPUs) that are coupled to storage devices including primary storage 1006 (typically a random access memory, or RAM), primary storage 1004 (typically a read only memory, or ROM). CPU 1002 may be of various types including microcontrollers and microprocessors such as programmable devices (e.g., CPLDs and FPGAs) and non-programmable devices such as gate array ASICs or general-purpose microprocessors. In the depicted embodiment, primary storage 1004 acts to transfer data and instructions uni-directionally to the CPU and primary storage 1006 is used typically to transfer data and instructions in a bidirectional manner. Both of these primary storage devices may include any suitable computer-readable media such as those described above. A mass storage device 1008 is also coupled bi-directionally to primary storage 1006 and provides additional data storage capacity and may include any of the computer-readable media described above. Mass storage device 1008 may be used to store programs, data and the like and is typically a secondary storage medium such as a hard disk. Frequently, such programs, data and the like are temporarily copied to primary memory 1006 for execution on CPU 1002. It will be appreciated that the information retained within the mass storage device 1008, may, in appropriate cases, be incorporated in standard fashion as part of primary storage 1004. A specific mass storage device such as a CD-ROM 1014 may also pass data uni-directionally to the CPU or primary storage.

CPU 1002 is also coupled to an interface 1010 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1002 optionally may be coupled to an external device such as a database or a computer or telecommunications network using an external connection as shown generally at 1012. With such a connection, it is contemplated that the CPU might receive information from the network, or might output information to the network in the course of performing the method steps described herein.

In one embodiment, a system such as computer system 1000 is used as a biological classification tool that employs gradient determination, thresholding, and/or shape characterization routines for analyzing image data for biological systems. System 1000 may also serve as various other tools associated with biological classification such as an image capture tool. Information and programs, including image files and other data files can be provided via a network connection 1012 for downloading by a researcher. Alternatively, such information, programs and files can be provided to the researcher on a storage device.

In a specific embodiment, the computer system 1000 is directly coupled to an image acquisition system such as an optical imaging system that captures images of cells or other biological features. Digital images from the image generating system are provided via interface 1012 for image analysis by system 1000. Alternatively, the images processed by system 1000 are provided from an image storage source such as a database or other repository of cell images. Again, the images are provided via interface 1012. Once in apparatus 1000, a memory device such as primary storage 1006 or mass storage 1008 buffers or stores, at least temporarily, digital images of the cells. In addition, the memory device may store the quantitative phenotypes that represent the points on the response path. The memory may also store various routines and/or programs for analyzing and presenting the data, including the elongation characterization and stimulus response paths. Such programs/routines may include programs for identifying edges, characterizing the shapes of such edges, performing path comparisons (e.g., distance or similarity calculations, as well as clustering and classification operations), principal component analysis, regression analyses, and for graphical rendering of the edge data.

### Other Embodiments

Although the above has generally described the present invention according to specific processes and apparatus, the present invention has a much broader range of applicability. For example, the present invention has been described in terms of characterizing biological image data based on a degree of elongation in biological features, but is not so limited. The elongation analyses of this invention may be employed outside the context of biological systems and images of cells. Of course, those of ordinary skill in the art will recognize other modifications and alternatives.

1. A method of characterizing cellular elongation in a population of cells, the method comprising:
(a) receiving image data showing signal intensity versus position in an image of the population of cells;
(b) determining values of a gradient in signal intensity at multiple positions in the image;
(c) identifying edges of cells in the image by using the values of the gradient determined in (b);
(d) determining whether individual edges identified in (c) are elongated; and
(e) characterizing the population of cells based on the determination in (d).

2. The method of claim 1, wherein the cellular elongation of the population of cells is characterized without segmenting the image data into regions of individual cells.

3. The method of claim 1, wherein the image was produced using at least one of phase contrast microscopy, Hoffman modulation contrast microscopy, differential interference contrast microscopy, and bright field microscopy.

4. The method of claim 1, wherein the population of cells comprises fungal cells.

5. The method of claim 1, wherein the image of the population of cells was obtained when at least some of the cells were alive.

6. The method of claim 1, further comprising:
exposing the population of cells to a stimulus prior to producing the image of the population of cells;
performing (a)-(e) on a control population of cells that has not been exposed to the stimulus; and
comparing the elongation of the edges in both populations of cells to gain information about the effect of the stimulus on the population of cells.

7. The method of claim 6, wherein the stimulus is a chemical compound.

8. The method of claim 1, further comprising:
treating the population of cells with a chemical compound in a first concentration prior to producing the image of the population of cells;
treating a second population of cells with the chemical compound in a second concentration;
performing (a)-(e) on a second population of cells; and
comparing the elongation of the edges in both populations of cells to gain information about the dose response of the chemical compound.

9. The method of claim 1, wherein the population of cells comprises fungal cells, and wherein characterizing the population of cells classifies a treatment of the fungal cells based on its ability to arrest progress through the cell cycle.

10. The method of claim 1, wherein determining values of the gradient in signal intensity comprises (i) determining first values of the gradient in a first direction along the image, and (ii) determining second values of the gradient in a second direction along the image.

11. The method of claim 10, wherein the first and second directions are substantially orthogonal to one another.

12. The method of claim 1, wherein identifying edges of cells in the image by using the values of the gradient comprises selecting pixels having values of the gradient that are greater than a threshold value.

13. The method of claim 12, further comprising calculating the threshold value from the image data using an adaptive threshold technique.

14. The method of claim 12, wherein selecting pixels having values of the gradient that are greater than a threshold value comprises selecting both pixels having positive gradient values that are greater than a positive value of the threshold and pixels having negative gradient values that are more negative than a negative value of the threshold.

15. The method of claim 1, wherein determining whether individual edges are elongated comprises determining values of a shape descriptor for individual ones of the edges.

16. The method of claim 15, wherein the shape descriptor specifies a degree of curvature in an edge.

17. The method of claim 16, wherein the shape descriptor is a circular variance in an edge.

18. The method of claim 1, wherein characterizing the population of cells comprises determining a fraction of edges or edge pixels in the image that are determined to be elongated.

19. A computer program product comprising a machine readable medium on which is provided program instructions for characterizing cellular elongation in a population of cells, wherein the program instructions comprise:
(a) code for determining values of a gradient in signal intensity at multiple positions in an image showing signal intensity versus position in an image of the population of cells;
(b) identifying edges of cells in the image by using the values of the gradient determined in (a);
(c) determining whether individual edges identified in (b) are elongated; and
(d) characterizing the population of cells based on the determination in (c).

20. The computer program product of claim 19, further comprising:
code for performing (a)-(d) on multiple images of different population of cells, some of which have been exposed to a stimulus and some of which have not been exposed to a stimulus; and
code for comparing the elongation of the edges in the populations of cells to gain information about the effect of the stimulus on the population of cells.

21. The computer program product of claim 20 wherein the stimulus is a chemical compound.

22. The computer program product of claim 19, wherein the code for determining values of the gradient in signal intensity comprises code for (i) determining first values of the gradient in a first direction along the image, and (ii) determining second values of the gradient in a second direction along the image.

23. The computer program product of claim 22, wherein the first and second directions are substantially orthogonal to one another.

24. The computer program product of claim 1, wherein the code for identifying edges of cells in the image by using the values of the gradient comprises code for selecting pixels having values of the gradient that are greater than a threshold value.

25. The computer program product of claim 24, further comprising code for calculating the threshold value from the image data using an adaptive threshold technique.

26. The computer program product of claim 24, wherein the code for selecting pixels having values of the gradient that are greater than a threshold value comprises code for selecting both pixels having positive gradient values that are greater than a positive value of the threshold and pixels having negative gradient values that are more negative than a negative value of the threshold.

27. The computer program product of claim 1, wherein the code for determining whether individual edges are elongated comprises code for determining values of a shape descriptor for individual ones of the edges.

28. The computer program product of claim 27, wherein the shape descriptor specifies a degree of curvature in an edge.

29. The computer program product of claim 28, wherein the shape descriptor is a circular variance in an edge.

30. The computer program product of claim 19, wherein the code for characterizing the population of cells comprises code for determining a fraction of edges in the image that are determined to be elongated or edge pixels belonging to elongated edges in the image.

31. A method of characterizing cellular elongation in a population of fungal cells, the method comprising:
(a) receiving image data showing signal intensity versus position in an image of the population of fungal cells;
(b) determining values of a gradient in signal intensity at individual pixels in the image, wherein the gradient values are determined in at least two directions in the image;
(c) identifying edges of cells in the image by comparing the values of the gradient determined in each of the at least two directions to threshold values, wherein individual ones of the identified edges comprise groups of pixels to one another proximate and wherein the edges define only portions of the boundaries of the fungal cells;
(d) determining curvature values for at least some of the edges to thereby characterize those edges as elongated or not; and
(e) characterizing the population of fungal cells based on the determination in (d).

32. The method of claim 31, wherein the population of fungal cells is characterized in (e) for the effect of a treatment on the fungal cells.

33. The method of claim 32, wherein the treatment is contact with a known or putative anti-fungal agent.

34. The method of claim 32, wherein a characterization produced in (e) indicates a likelihood that the treatment impacts progress through the cell cycle in at least some of the fungal cells.

35. The method of claim 32, wherein a characterization produced in (e) indicates a likelihood that the treatment impacts the transition between filamentous and non-filamentous growth forms in at least some of the fungal cells.

36. The method of claim 32, further comprising:
repeating (a)-(e) for each of a plurality of fungal cell populations, wherein each population is exposed to a different level of the treatment; and
comparing characterizations produced in (e) to produce a dose response curve for the treatment.

37. The method of claim 36, wherein the dose response curve is used to assess potency of the treatment.

38. The method of claim 31, wherein characterizing the population of fungal cells comprises determining a fraction of edges in the image that are determined to be elongated or edge pixels belonging to elongated edges in the image.

39. The method of claim 31, wherein the edges identified in (c) are identified without first identifying individual cells in the image.

40. The method of claim 31, wherein the image was produced using at least one of phase contrast microscopy, Hoffman modulation contrast microscopy, differential interference contrast microscopy, and bright field microscopy.

41. A computer program product comprising a machine readable medium on which is provide program instructions for characterizing cellular elongation in a population of fungal cells, the program instructions comprising:
(a) code for receiving image data showing signal intensity versus position in an image of the population of cells;
(b) code for determining values of a gradient in signal intensity at individual pixels in the image, wherein the gradient values are determined in at least two directions in the image;
(c) code for identifying edges of cells in the image by comparing the values of the gradient determined in each of the at least two directions to threshold values, wherein individual ones of the identified edges comprise groups of pixels to one another proximate and wherein the edges define only portions of the boundaries of the cells;
(d) code for determining curvature values for at least some of the edges to thereby characterize those edges as elongated or not; and
(e) code for characterizing the population of cells based on the determination in (d).

42. The method of claim 41, wherein the code for characterizing the population of fungal cells comprises code for determining a fraction of edges in the image that are determined to be elongated or edge pixels belonging to elongated edges in the image.

43. An apparatus for characterizing cellular elongation in a population of cells, the apparatus comprising:
a processor;
memory; and
an interface configured for receiving image data showing signal intensity versus position in an image of the population of cells, wherein at least one of the processor and memory is configured to
(i) determine values of a gradient in signal intensity at multiple positions in the image;
(ii) identify edges of cells in the image by using the values of the gradient determined in (i); and
(iii) determine whether individual edges identified in (ii) are elongated.

44. The apparatus of claim 43, further comprising an imaging device in communication with the interface, wherein the image device employs at least one of phase contrast microscopy, Hoffman modulation contrast microscopy, differential interference contrast microscopy, and bright field microscopy.

45. The apparatus of claim 43, wherein at least one of the processor and memory is configured to determine values of the gradient in signal intensity by (i) determining first values of the gradient in a first direction along the image, and (ii) determining second values of the gradient in a second direction along the image.

46. The apparatus of claim 45, wherein the first and second directions are substantially orthogonal to one another.

47. The apparatus of claim 43, wherein at least one of the processor and memory is configured to identify edges of cells in the image by using the values of the gradient by selecting pixels having values of the gradient that are greater than a threshold value.

48. The apparatus of claim 47, wherein at least one of the processor and memory is configured to calculate the threshold value from the image data using an adaptive threshold technique.

49. The apparatus of claim 47, wherein selecting pixels having values of the gradient that are greater than a threshold value comprises selecting both pixels having positive gradient values that are greater than a positive value of the threshold and pixels having negative gradient values that are more negative than a negative value of the threshold.

50. The apparatus of claim 43, wherein at least one of the processor and memory is configured to determine whether individual edges are elongated by determining values of a shape descriptor for individual ones of the edges.

51. The apparatus of claim 50, wherein the shape descriptor specifies a degree of curvature in an edge.
